(19) United States
(12) Patent Application Publication
(10) Pub. No.: US 2021/0007612 A1
(43) Pub. Date: Jan. 14, 2021

(54) ASSISTING SYSTEM, ASSISTING METHOD, ASSISTING PROGRAM, AND RECORDING MEDIUM RECORDING AN ASSISTING PROGRAM

(71) Applicant: TERUMO KABUSHIKI KAISHA, Tokyo (JP)

(72) Inventors: Shin MAKI, Ebina-shi (JP); Tomoko UEMURA, Hadano-shi (JP)

(73) Assignee: TERUMO KABUSHIKI KAISHA, Tokyo (JP)

(21) Appl. No.: 17/032,245

(22) Filed: Sep. 25, 2020

Related U.S. Application Data
(63) Continuation of application No. PCT/JP2019/012814, filed on Mar. 26, 2019.

(30) Foreign Application Priority Data
Mar. 26, 2018 (JP) 2018-058065

(51) Int. Cl. A61B 5/0205; A61B 5/02; A61B 5/00

(52) U.S. Cl. CPC A61B 5/02055; A61B 5/7264; A61B 5/02007

(57) ABSTRACT

An assisting system for assisting in predicting the exacerbation of a heart failure, including: a data acquiring section configured to acquire chronological data of amounts of congestion in at least a portion of the body of a patient and chronological data of a blood flow rate in a limb of the patient; an assessing section configured to chronically calculate an overall assessment value depending on the levels of the amounts of congestion and the blood flow rate in the limb, using the chronological data of the amounts of congestion and the chronological data of the blood flow rate; and a predicting section configured to predict a transition of the overall assessment value using the chronologically calculated overall assessment value.

### CROSS-REFERENCES TO RELATED APPLICATIONS

This application is a continuation of International Application No. PCT/JP2019/012814 filed on Mar. 26, 2019, which claims priority to Japanese Application No. 2018-058065 filed on Mar. 26, 2018, the entire content of both of which is incorporated herein by reference.

### FIELD

The present disclosure generally relates to an assisting system, an assisting method, an assisting program, and a recording medium recording an assisting program for assisting in predicting the exacerbation of a heart failure.

### BACKGROUND DISCUSSION

Heart failure refers to a disease in which a heart malfunctions as a pump, leading to a reduction in cardiac output, congestion in the lungs and the systemic vein system, etc. Patients who have suffered heart failure are often liable to have the symptom recur gradually and become hospitalized again after a period of remission.

One approach for diagnosing heart failures is Nohria-Stevenson classification (see “2013 ACCF/AHA Guideline for the Management of Heart Failure,” [online], American College of Cardiology Foundation (ACCF), American Heart Association (AHA), [retrieved Feb. 20, 2018, The Internet <URL: http://circ.ahajournals.org/content/128/16/e240> below) that classifies disease states of heart failure into four categories. According to the diagnostic process using Nohria-Stevenson classification, the doctor determines, from a physical examination of a patient, whether or not there is congestion in the body of the patient and whether or not there is hypoperfusion, for example, whether or not the patient's heart is delivering sufficient blood to body tissues, and classifies the patient's disease state depending on the determination.

Diagnoses using Nohria-Stevenson classification are performed on the basis of doctor's findings and hence is governed by doctor's experiences. For example, a general internist at a clinic will generally perform a follow-up on a patient who has been treated by a heart-failure specialist and has achieved remission. However, the general internist may fail, for example, to diagnose the patient properly due to a lack of experience as a heart-failure specialist. When the patient's condition takes a turn for the worse, the patient may have to be sent to a medical organization to which a specialist in heart failure practices. It has been difficult for doctors to predict the exacerbation of a heart failure on the basis of Nohria-Stevenson classification.

### SUMMARY

An assisting system, an assisting method, an assisting program, and a recording medium recording an assisting program are disclosed that are capable of assisting in predicting the exacerbation of a heart failure on the basis of Nohria-Stevenson classification.

In accordance with an aspect, an assisting system is disclosed for assisting in predicting the exacerbation of a heart failure, including a data acquiring section configured to acquire chronological data of amounts of congestion in at least a portion of the body of a patient and chronological data of a blood flow rate in a limb of the patient, an assessing section configured to chronically calculate an overall assessment value depending on the levels of the amounts of congestion and the blood flow rate in the limb, using the chronological data of the amounts of congestion and the chronological data of the blood flow rate, and a predicting section configured to predict a transition of the overall assessment value using the chronologically calculated overall assessment value.

In accordance with another aspect, an assisting method is disclosed for assisting in predicting the exacerbation of a heart failure, including acquiring chronological data of amounts of congestion in at least a portion of the body of a patient and chronological data of a blood flow rate in a limb of the patient, chronologically calculating an overall assessment value depending on the levels of the amounts of congestion and the blood flow rate in the limb, using the chronological data of the amounts of congestion and the chronological data of the blood flow rate, and predicting a transition of the overall assessment value using the chronologically calculated overall assessment value.

In accordance with an aspect, a non-transitory computer-readable medium for assisting in predicting exacerbation of a heart failure is disclosed, the non-transitory computer-readable medium having instructions operable to cause one or more processors to perform operations comprising: acquiring chronological data of amounts of congestion in at least a portion of the body of a patient and chronological data of a blood flow rate in a limb of the patient, a procedure for chronologically calculating an overall assessment value depending on the levels of the amounts of congestion and the blood flow rate in the limb, using the chronological data of the amounts of congestion and the chronological data of the blood flow rate, and a procedure for predicting a transition of the overall assessment value using the chronologically calculated overall assessment value.

According to the present disclosure, it is possible to assist in predicting the exacerbation of a heart failure on the basis of Nohria-Stevenson classification.

### BRIEF DESCRIPTION OF THE DRAWINGS

FIG. 1 is a schematic diagram illustrating a general outline of an assisting system according to an exemplary embodiment of the present disclosure.

FIG. 2 is a table illustrating Nohria-Stevenson classification.

FIG. 3 is a block diagram of the hardware configuration of a server included in the assisting system according to the exemplary embodiment.

FIG. 4 is a block diagram of the functional configuration of a central processing unit (CPU) of the server included in the assisting system according to the exemplary embodiment.

FIG. 5A is a diagram illustrating data handled by the assisting system according to the exemplary embodiment.

FIG. 5B is a diagram illustrating a data processing process of the assisting system according to the exemplary embodiment.

FIG. 6 is a diagram illustrating the manner in which the assisting system according to the exemplary embodiment predicts the exacerbation of a heart failure.

FIG. 7 is a flowchart of an assisting method according to the exemplary embodiment.

### DETAILED DESCRIPTION

Set forth below with reference to the accompanying drawings is a detailed description of embodiments of an assisting system, an assisting method, an assisting program, and a recording medium recording an assisting program for assisting in predicting the exacerbation of a heart failure representing examples of the inventive assisting system, assisting method, assisting program, and recording medium recording an assisting program for assisting in predicting the exacerbation of a heart failure. Note that since embodiments described below are exemplary examples of the present disclosure, although various technically exemplary limitations are given, the scope of the present disclosure is not limited to the embodiments unless otherwise specified in the following descriptions. Identical parts in some figures of the drawings are denoted by identical reference characters, and their redundant description will be omitted below. Some dimensional ratios in the drawings are illustrated exaggerated for illustrative purposes and may differ from actual dimensional ratios.

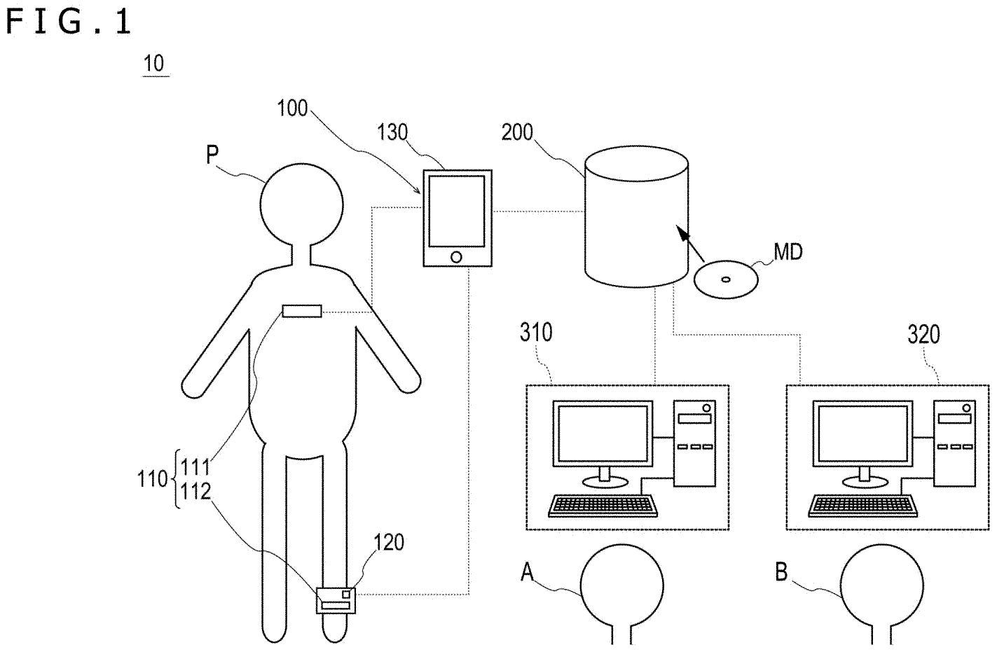

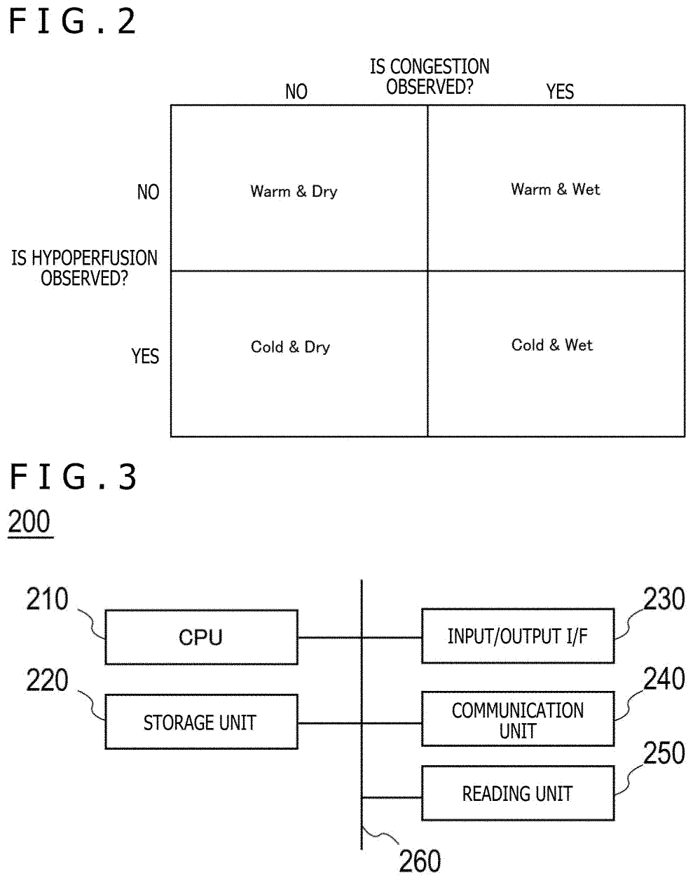

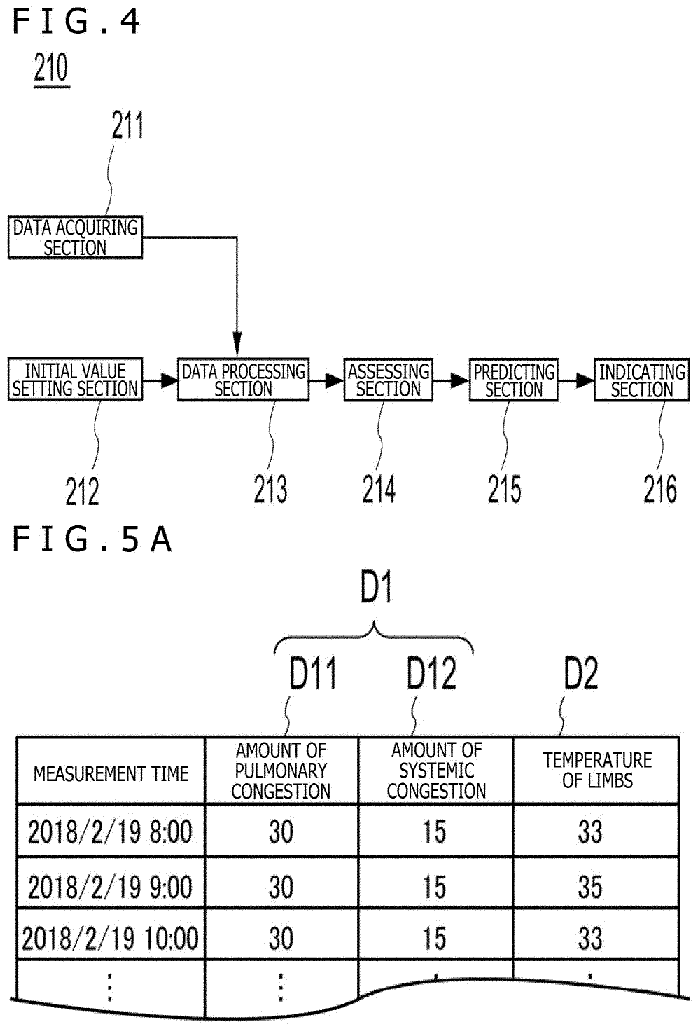

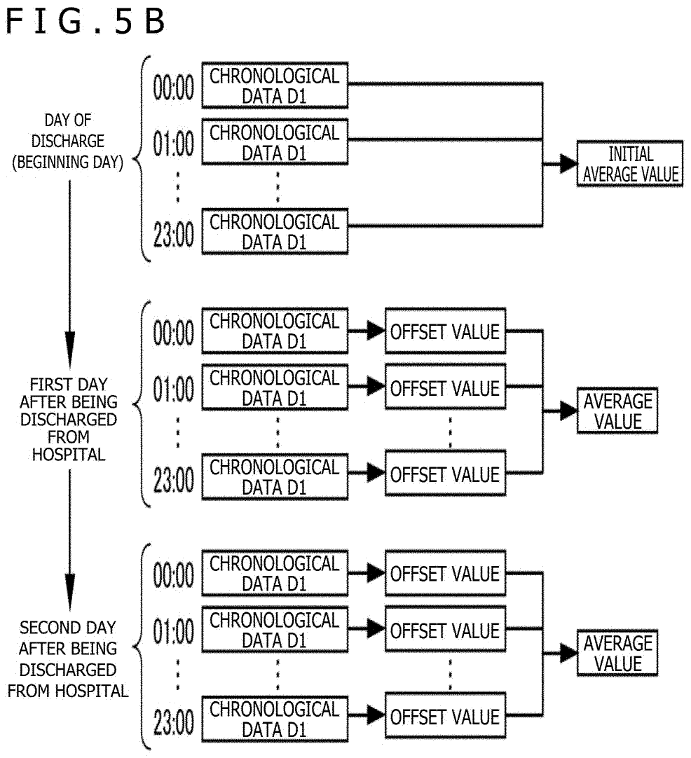

FIG. 1 schematically illustrates a general outline of an assisting system 10 according to the exemplary embodiment of the present disclosure. FIG. 2 illustrates Nohria-Stevenson classification. FIGS. 3 and 4 illustrate details of a server included in the assisting system 10 according to the exemplary embodiment. FIGS. 5A through 6 illustrate data handled by the assisting system 10 according to the exemplary embodiment.

As illustrated in FIG. 1, according to the present embodiment, the assisting system 10 is configured as a system for assisting a patient P and doctors A, B, etc., each as a user in predicting the exacerbation of a heart failure of the patient P according to Nohria-Stevenson classification. The assisting system 10 is not limited to any particular applications, and may be used, for example, in a situation where a general internist B at a clinic does a follow-up on a patient P who has been treated by a heart-failure specialist A and has achieved remission, and refers the patient P to the specialist A for treatment depending on the condition of the patient P.

As illustrated in FIG. 2, Nohria-Stevenson classification classifies disease states of heart failure into four categories on the basis of whether or not there is congestion in the body of the patient P and whether or not there is hypoperfusion, i.e., whether or not the patient's heart is delivering sufficient blood to body tissues. The first disease state represents a group Warm & Dry (upper left in FIG. 2) with no congestion and no hypoperfusion. The second disease state represents a group Warm & Wet (upper right in FIG. 2) with congestion and no hypoperfusion. The third disease state represents a group Cold & Dry (lower left in FIG. 2) with no congestion and hypoperfusion. The fourth disease state represents a group Cold & Wet (lower right in FIG. 2) with congestion and hypoperfusion. The group Warm & Dry indicates that the patient P is in a good condition, whereas the groups Warm & Wet, Cold & Dry, and Cold & Wet, particularly the group Cold & Wet, indicate that the patient P is in worse conditions. Thus, Nohria-Stevenson classification classifies disease states of heart failure by comprehensively assessing the level of congestion and the level of hypoperfusion of the patient P.

The assisting system 10 will generally be described below with reference to FIG. 1. The assisting system 10 can include a measuring unit 100 for measuring an amount of congestion in at least a portion of the body of the patient P and a blood flow rate in a limb of the patient P, and a server 200 connected through a network, indicated by the broken lines, to the measuring unit 100 and operating terminals 310 and 320 of the doctors A and B, for sending data to and receiving data from the measuring unit 100 and the operating terminals 310 and 320. The components of the assisting system 10 will be described in detail below.

Measuring Unit

The measuring unit 100 has a congestion measuring unit 110 for measuring an amount of congestion in at least a portion of the body of the patient P, a blood flow rate measuring unit 120 for measuring a blood flow rate in a patient's limb, and a control unit 130 for controlling operation of the congestion measuring unit 110 and the blood flow rate measuring unit 120. These components of the measuring unit 100 will be described in detail below.

In accordance with an aspect, the measuring units 110 and 120 are constructed as wearable devices. The measuring units 110 and 120 are configured to obtain measurements at any timings that are not limited to particular timings. For example, the measuring units 110 and 120 may obtain measurements at time intervals ranging from every one minute to every one hour while the measuring units 110 and 120 are being worn by the patient P. The timings at which the measuring units 110 and 120 make measurements may be set to suitable timings depending on the condition of the patient P. In accordance with an aspect, the measuring units 110 and 120 may not be constructed as wearable devices.

According to the present exemplary embodiment, the congestion measuring unit 110 includes a pulmonary congestion measuring unit 111 for measuring an amount of pulmonary congestion of the patient P and a systemic congestion measuring unit 112 for measuring an amount of systemic congestion of the patient P. The pulmonary congestion measuring unit 111 is not limited to any particular device insofar as the pulmonary congestion measuring unit 111 is capable of measuring an amount of pulmonary congestion of the patient P. However, the pulmonary congestion measuring unit 111 may be a device, for example, capable of measuring a water content of the lungs of the patient P using a thoracic impedance, ultrasonic waves, a microphone, a percutaneous arterial oxygen saturation, a local tissue oxygen saturation, or the like. The systemic congestion measuring unit 112 is not limited to any particular device insofar as the systemic congestion measuring unit 112 is capable of measuring an amount of systemic congestion of the patient P. However, the systemic congestion measuring unit 112 may be, for example, a device capable of measuring a dropsical swelling of a limb, illustrated as a leg, of the patient P by measuring the circumference of the limb of the patient P or the bioimpedance of the limb of the patient P.

In accordance with an exemplary embodiment, the blood flow rate measuring unit 120 is a temperature sensor capable of measuring a change in the body surface temperature, for example, a cold extremity, due to a change in the blood flow rate in the limb, for example, the leg, of the patient P. However, the blood flow rate measuring unit 120 is not limited to any particular device insofar as the blood flow rate measuring unit 120 is capable of directly or indirectly measuring a blood flow rate in the limb of the patient P. For example, the blood flow rate measuring unit 120 may be, for example, a deice such as a camera capable of measuring a color change due to a change in the oxygen rate, for example, a change in the blood flow rate, in the limb of the patient P. Alternatively, the blood flow rate measuring unit 120 may measure both the temperature and color of the limb of the patient P.

In accordance with an aspect, the control unit 130 is connected to the measuring units 110 and 120 through, for example, a wireless communication network, indicated by the broken lines. The control unit 130 controls measuring operation of the measuring units 110 and 120, acquires measured data from the measuring units 110 and 120, and sends the acquired measured data to the server 200.

Server

As illustrated in FIG. 3, the server 200 can include a CPU 210, a storage unit 220, an input/output interface (I/F) 230, a communication unit 240, and a reading unit 250. The CPU 210, the storage unit 220, the input/output I/F 230, the communication unit 240, and the reading unit 250 are connected to a bus 260 and exchange data, etc. through the bus 260. These components of the server 200 will be described below.

The CPU 210 controls the other components and performs various arithmetic processing operations according to various programs stored in the storage unit 220.

The storage unit 220 can include a read only memory (ROM) for storing various programs and various kinds of data, a random access memory (RAM) as a working area for temporarily storing programs and data, and a hard disk or the like for storing various programs including an operating system and various kinds of data. The storage unit 220 stores various kinds of data and programs such as assisting programs.

The communication unit 240 is an interface for communicating with the measuring unit 100 and the operating terminals 310 and 320 of the doctors A, B, etc.

The reading unit 250 reads assisting programs, etc. recorded in a computer-readable recording medium MD (see FIG. 1). The computer-readable recording medium MD is not limited to any particular medium, for example, the computer-readable recording medium MD may be an optical disk such as a compact disc read-only memory (CD-ROM) or a digital versatile disc read-only memory (DVD-ROM), a universal serial bus (USB) memory, a secure digital (SD) memory card, or the like. The reading unit 250 is not limited to any particular device, for example, the reading unit 250 may be a CD-ROM drive, a DVD-ROM drive, or the like.

Next, main functions of the CPU 210 will be described below.

The CPU 210 functions as a data acquiring section 211, an initial value setting section 212, a data processing section 213, an assessing section 214, a predicting section 215, and an indicating section 216 corresponding to “result indicating section” and “accuracy indicating section,” by executing the assisting programs stored in the storage unit 220. These sections will hereinafter be described below.

First, the data acquiring section 211 will be described below.

According to the present exemplary embodiment, as illustrated in FIG. 5A, the data acquiring section 211 acquires, from the measuring unit 100, chronological data D1 of amounts of congestion in at least a portion of the body of the patient P (hereinafter simply referred to as “chronological data D1 of amounts of congestion”) and chronological data D2 of a blood flow rate in the limb of the patient P.

According to the present embodiment, the chronological data D1 of amounts of congestion includes chronological data D11 of an amount of pulmonary congestion and chronological data D12 of an amount of systemic congestion.

According to the present exemplary embodiment, the chronological data D2 of a blood flow rate in the limb can include chronological data of a temperature of the limb.

As illustrated in FIG. 5A, the acquired chronological data D1 of amounts of congestion and the acquired chronological data D2 of a blood flow rate in the limb are stored in the storage unit 220 in association with measurement times.

Next, the initial value setting section 212 will be described below.

According to the present exemplary embodiment, the initial value setting section 212 prompts the user to specify an initial value for an amount of congestion depending on the level of congestion of the patient P on the day when the measuring unit 100 starts measurements. The initial value setting section 212 sets an initial value for an amount of congestion to the value specified by the user.

Specifically, in a case where the measuring unit 100 starts measurements from the day (hereinafter referred to as “day of discharge”) when the patient P is discharged from a medical organization to which the heart-failure specialist A belongs, providing the patient P has completely recovered from pulmonary congestion and systemic congestion on the day of discharge, the doctor A as the user specifies 0 (zero) for an amount of pulmonary congestion and an amount of systemic congestion. Alternatively, if the patient P has not sufficiently recovered from pulmonary congestion and systemic congestion on the day of discharge, the doctor A as the user specifies values depending on the levels of the amount of pulmonary congestion and the amount of systemic congestion of the patient P on the day of discharge as initial values for an amount of pulmonary congestion and an amount of systemic congestion.

Next, the data processing section 213 will be described below.

The data processing section 213 pretreats the chronological data D1 and D2 before the assessing section 214 to be described later calculates a congestion assessment value C1 and a blood flow assessment value C2.

As illustrated in FIG. 5B, on the day when the measuring unit 100 starts measurements, for example, on the day of discharge, the data processing section 213 calculates an average value of the chronological data D1 of the amounts of congestion measured at predetermined timings, for example, at time intervals ranging from every one minute to every one hour. The calculated value will hereinafter simply be referred to as “initial average value of the chronological data D1 of the amounts of congestion.”

Next, the data processing section 213 calculates a value by subtracting the initial average value of the chronological data D1 of the amounts of congestion from the chronological data D1 of the amounts of congestion measured after the day when the measuring unit 100 has started measurements, for example, after the day of discharge, and adding the initial values for the amounts of congestion set by the initial value setting section 212 to the difference. The calculated value will hereinafter simply be referred to as “offset value of the chronological data D1 of the amounts of congestion.”

Next, the data processing section 213 calculates an average value representing an average of offset values of the chronological data D1 of the amounts of congestion per predetermined period, for example, per day. The calculated average value will hereinafter be referred to as “average value of the chronological data D1 of the amounts of congestion.” The average value of the chronological data D1 of the amounts of congestion can include an average value of the chronological data D11 of the amount of pulmonary congestion and an average value of the chronological data D12 of the amount of systemic congestion. The average value of the chronological data D1 of the amounts of congestion thus represents a change from the initial value of the amount of congestion specified by the doctor.

The data processing section 213 calculates an average value of the chronological data D2 of the blood flow rate in the limb measured per predetermined period, for example, per day. The calculated value will hereinafter simply be referred to as “average value of the chronological data D2 of the blood flow rate in the limb.”

The way in which the data processing section 213 pretreats the chronological data D1 and D2 is not limited to the above process. Instead of calculating average values of the chronological data D1 and D2 measured per predetermined period, the data processing section 213 may calculate median values, minimum values, maximum values, or the like of the chronological data D1 and D2 measured per predetermined period. The assessing section 214 to be described below may then calculate an assessment value C1 of the congestion and an assessment value C2 of the blood flow using the median values, the minimum values, the maximum values, or the like of the chronological data D1 and D2.

Next, the assessing section 214 will be described below.

The assessing section 214 chronologically calculates a first overall assessment value V1 depending on the levels of the amount of pulmonary congestion and the blood flow rate in the limb of the patient P, using the average value of the chronological data D11 of the amount of pulmonary congestion and the average value of the chronological data D2 of the blood flow rate in the limb. Furthermore, the assessing section 214 chronologically calculates a second overall assessment value V2 depending on the levels of the amount of systemic congestion and the blood flow rate in the limb of the patient P, using the average value of the chronological data D12 of the amount of systemic congestion and the average value of the chronological data D2 of the blood flow rate in the limb. The first overall assessment value V1 represents the level of the exacerbation of a left cardiac failure, and the second overall assessment value V2 represents the level of the exacerbation of a right cardiac failure. A process of calculating the first overall assessment value V1 and the second overall assessment value V2 will hereinafter be described below.

The assessing section 214 chronologically calculates a pulmonary congestion assessment value C11 depending on the level of pulmonary congestion, using the average value of the chronological data D11 of the amount of pulmonary congestion. According to the present exemplary embodiment, the assessing section 214 classifies the amount of pulmonary congestion into a plurality of stages, for example, values of thoracic bioimpedance, and determines pulmonary congestion assessment values C11 as points respectively for the stages, as indicated by Table 1 below. According to the present exemplary embodiment, the assessing section 214 classifies the amount of pulmonary congestion into eleven (11) stages and gives pulmonary congestion assessment values C11 as 0 through 10 points respectively to the states. The larger the amount of pulmonary congestion is, the larger the assessment values C11 are. For example, if the average value of the chronological data D11 of the amount of pulmonary congestion, for example, the thoracic impedance, on a day is 45Ω (ohms), then the corresponding pulmonary congestion assessment value C11 is indicated as 9 points. The values illustrated in Table 1 are by way of example only, and the pulmonary congestion assessment values C11 are not limited to the values in Table 1.

TABLE 1

| Amount Z of pulmonary congestion | Pulmonary congestion |
|---|---|
| (thoracic bioimpedance [Ω]) | evaluation value C11 [points] |
| 0 ≤ Z < 5 | 0 |
| 5 ≤ Z < 10 | 1 |
| 10 ≤ Z < 15 | 2 |
| 15 ≤ Z < 20 | 3 |
| 20 ≤ Z < 25 | 4 |
| 25 ≤ Z < 30 | 5 |
| 30 ≤ Z < 35 | 6 |
| 35 ≤ Z < 40 | 7 |
| 40 ≤ Z < 45 | 8 |
| 45 ≤ Z < 50 | 9 |
| 50 ≤ Z | 10 |

In accordance with an exemplary embodiment, instead of calculating a pulmonary congestion evaluation value C11 using Table 1, the assessing section 214 may calculate a pulmonary congestion evaluation value C11 according to a conversion formula that converts the average value of the chronological data D11 of the amount of pulmonary congestion into a pulmonary congestion evaluation value C11.

The assessing section 214 chronologically calculates a systemic congestion assessment value C12 depending on the level of systemic congestion, using the average value of the chronological data D12 of the amount of systemic congestion. According to the present exemplary embodiment, the assessing section 214 classifies the amount of systemic congestion into a plurality of stages, for example, values of the circumference of the limb, and determines systemic congestion assessment values C12 as points respectively for the stages, as indicated by Table 2 below. According to the present exemplary embodiment, the assessing section 214 classifies the amount of systemic congestion into eleven (11) stages and gives systemic congestion assessment values C12 as 0 through 10 points respectively to the states. The larger the amount of systemic congestion is, the larger the systemic congestion assessment values C12 are. For example, if the average value of the chronological data D12 of the amount of systemic congestion, for example, the circumference of the limb, on a day is 45 mm, then the corresponding systemic congestion assessment value C12 is indicated as 9 points. The values illustrated in Table 2 are by way of example only, and the systemic congestion assessment values C12 are not limited to the values in Table 2.

TABLE 2

| Amount R of systemic congestion | Systemic congestion evaluation |
|---|---|
| (circumference of limb [mm]) | value C12 [points] |
| 0 ≤ R < 5 | 0 |
| 5 ≤ R < 10 | 1 |
| 10 ≤ R < 15 | 2 |
| 15 ≤ R < 20 | 3 |
| 20 ≤ R < 25 | 4 |
| 25 ≤ R < 30 | 5 |
| 30 ≤ R < 35 | 6 |
| 35 ≤ R < 40 | 7 |
| 40 ≤ R < 45 | 8 |
| 45 ≤ R < 50 | 9 |
| 50 ≤ R | 10 |

In accordance with an exemplary embodiment, instead of calculating a systemic congestion evaluation value C12 using Table 2, the assessing section 214 may calculate a systemic congestion evaluation value C12 according to a conversion formula that converts the average value of the chronological data D12 of the amount of systemic congestion into a systemic congestion evaluation value C12, as indicated by the following Equation 1, for example:

Math 1

Systemic congestion assessment value C12=average value of chronological data D11 of the amount of systemic congestion(circumference of limb [mm])÷5 (Equation 1)

The assessing section 214 chronologically calculates a blood flow assessment value depending on the level of a blood flow rate in the limb, using the average value of the chronological data D2 of the blood flow rate in the limb. According to the present embodiment, the assessing section 214 classifies the blood flow rate in the limb into a plurality of stages, for example, values of the temperature of the limb, and determines blood flow assessment values C2 as points respectively for the stages, as indicated by Table 3 below. According to the present embodiment, the assessing section 214 classifies the blood flow rate in the limb into 11 stages and gives blood flow assessment values C2 as 0 through 10 points respectively to the states. The smaller the blood flow rate in the limb is, the larger the blood flow assessment values C2 are. For example, if the average value of the chronological data D2 of the blood flow rate in the limb on a day is 24° C., then the corresponding blood flow assessment value C2 is indicated as 9 points. The values illustrated in Table 3 are by way of example only, and the blood flow assessment values C2 are not limited to the values in Table 3.

TABLE 3

| Blood flow rate T in the limb | Blood flow evaluation |
|---|---|
| (temperature of the limb [° C.]) | value C2 [points] |
| 33 ≤ T | 0 |
| 32 ≤ T < 33 | 1 |
| 31 ≤ T < 32 | 2 |
| 30 ≤ T < 31 | 3 |
| 29 ≤ T < 30 | 4 |
| 28 ≤ T < 29 | 5 |
| 27 ≤ T < 28 | 6 |
| 26 ≤ T < 27 | 7 |
| 25 ≤ T < 26 | 8 |
| 24 ≤ T < 25 | 9 |
| T < 24 | 10 |

In accordance with an exemplary embodiment, instead of calculating a blood flow evaluation value C2 using Table 3, the assessing section 214 may calculate a blood flow evaluation value C2 according to a conversion formula that converts the average value of the chronological data D2 of the blood flow rate into a blood flow evaluation value C2, as indicated by the following Equation 2, for example.

Math 2

Blood flow assessment value C2=33−average value of the chronological data D2 of the blood flow rate(temperature of the limb[° C.]) (Equation 2)

The assessing section 214 substitutes the calculated pulmonary congestion assessment value C11 and the calculated blood flow evaluation value C2 for those in the equation (3) below, thereby calculating the first overall assessment value V1 depending on the levels of the amount of pulmonary congestion and the blood flow rate in the limb. According to the present embodiment, the first overall assessment value V1 can take a maximum value of 20 points and a minimum value of 0 point. The larger the first overall assessment value V1 is, the worse the left cardiac failure tends to become.

Math 3

First overall assessment value V1=W₁₁×pulmonary congestion assessment value C11+W12×blood flow assessment value C2 (Equation 3)

W₁₁: weighting coefficient for the pulmonary congestion assessment value

W₁₂: weighting coefficient for the blood flow assessment value

The weighting coefficients W₁₁ and W₁₂ are not limited to any particular values, but may be set to values within a range of their sum up to 2 depending on the tendency of each patient to develop the symptoms of pulmonary congestion and hypoperfusion. For example, the weighting coefficient W₁₁ may be set to a value larger than the weighting coefficient W₁₂ for those patients who are more likely to develop the symptom of pulmonary congestion than the symptom of a reduction in the blood flow rate in the limbs. For example, the weighting coefficient W₁₂ may be set to a value larger than the weighting coefficient W₁₁ for those patients who are more likely to develop the symptom of a reduction in the blood flow rate in the limbs than the symptom of pulmonary congestion. For example, the weighting coefficients W₁₁ and W₁₂ may be set to 1 for those patients who equally tend to develop the symptom of pulmonary congestion and the symptom of a reduction in the blood flow rate in the limbs. The weighting coefficients W₁₁ and W₁₂ may be set to values specified by the doctors A and B as the users or may be automatically set to values by the assessing section 214 from the values of the chronological data D11 of the amount of pulmonary congestion and the chronological data D2 of the blood flow rate in the limb, electronic clinical records, etc.

The assessing section 214 substitutes the calculated systemic congestion assessment value C12 and the blood flow assessment value C2 for those in the equation (4) below, thereby calculating the second overall assessment value V2 depending on the levels of the amount of systemic congestion and the blood flow rate in the limb. According to the present exemplary embodiment, the second overall assessment value V2 can take a maximum value of 20 points and a minimum value of 0 point. The larger the second overall assessment value V2 is, the worse the right cardiac failure tends to become.

Math 4

Second overall assessment value V2=W₂₁×systemic congestion assessment value C12+W₂₂×blood flow assessment value C2 (Equation 4)

W₂₁: weighting coefficient for the systemic congestion assessment value

W₂₂: weighting coefficient for the blood flow assessment value

The weighting coefficients W₂₁ and W₂₂ are not limited to any particular values, but may be set to values within a range of their sum up to 2 depending on the tendency of the patient P to develop the symptoms of systemic congestion and hypoperfusion. For example, the weighting coefficient W₂₁ may be set to a value larger than the weighting coefficient W₂₂ for those patients who are more likely to develop the symptom of systemic congestion than the symptom of a reduction in the blood flow rate in the limbs. For example, the weighting coefficient W₂₂ may be set to a value larger than the weighting coefficient W₂₁ for those patients who are more likely to develop the symptom of a reduction in the blood flow rate in the limbs than the symptom of systemic congestion. For example, the weighting coefficients W₁₁ and W₁₂ may be set to 1 for those patients who equally tend to develop the symptom of systemic congestion and the symptom of a reduction in the blood flow rate in the limbs. The weighting coefficients W₂₁ and W₂₂ may be set to values specified by the doctors A and B as the users or may be automatically set to values by the assessing section 214 from the values of the chronological data D12 of the amount of systemic congestion and the chronological data D2 of the blood flow rate in the limb, electronic clinical records, etc.

Next, the predicting section 215 will be described below.

The predicting section 215 predicts transitions of the first overall assessment value V1 and the second overall assessment value V2 using chronological sequences of the first overall assessment value V1 and the second overall assessment value V2. A process of predicting transitions of the first overall assessment value V1 and the second overall assessment value V2 will hereinafter be described below.

According to the present embodiment, as illustrated in FIG. 6, the predicting section 215 determines a first approximate formula F1 that approximates time-dependent changes in the first overall assessment value V1, indicated by solid circles, during a period t1 through t2 that goes back a predetermined period Δt from a latest measurement time t2 for the chronological data D1 and D2. Furthermore, the predicting section 215 determines a second approximate formula F2 that approximates time-dependent changes in the second overall assessment value V2, indicated by solid squares, during the period t1 through t2 that goes back the predetermined period Δt from the latest measurement time t2 for the chronological data D1 and D2. In FIG. 6, the first approximate formula F1 and the second approximate formula F2 are illustrated as linear functions, for example. However, the first approximate formula F1 and the second approximate formula F2 are not limited to linear functions, and may be any of functions of n-th degree including quadratic functions, exponential functions, or the like. For example, the predicting section 215 may apply approximate formulas of plural kinds to chronological data of the first overall assessment value V1 and chronological data of the second overall assessment value V2 and adopt approximate formulas that are of the highest approximation accuracy.

In accordance with an exemplary embodiment, the way in which the predicting section 215 determines the approximate formulas F1 and F2 is not limited to any particular process. For example, the predicting section 215 may employ a regression analysis process such as a least squares method. Moreover, the predicting section 215 may calculate a coefficient of determination as representing the accuracy of determined approximate formulas. Furthermore, in a case where the approximate formulas F1 and F2 are linear functions, the predicting section 215 may calculate gradients at two points that are next to each other during the period t1 through t2 of the approximate formulas F1 and F2 in the graph of FIG. 6 and calculate average values of the calculated gradients as the gradients of the approximate formulas F1 and F2.

As illustrated in FIG. 6, the predicting section 215 predicts a first timing t3 at which the first overall assessment value V1 will reach a threshold value Vth, using the first approximate formula F1. The predicting section 215 predicts a second timing t4 at which the second overall assessment value V2 will reach the threshold value Vth, using the second approximate formula F2. The threshold value Vth may be set to a value representing a predetermined level of exacerbation that a heart failure, for example, a left cardiac failure and a right cardiac failure, has reached. According to the present exemplary embodiment, the threshold value Vth is set to a level where the patient needs to be treated in hospital. However, the threshold value Vth may be set to a level where the patient needs to be examined in any of various medical organizations, such as a level where the patient needs to be examined by a general internist or a level where the patient needs to be examined by a specialist. Furthermore, the predicting section 215 may establish a plurality of threshold values Vth corresponding to a plurality of levels such as a level where the patient needs to be examined by a general internist, a level where the patient needs to be examined by a specialist, and a level where the patient needs to be hospitalized, and may predict timings at which the first overall assessment value V1 or the second overall assessment value V2 will reach the threshold value Vth. In FIG. 6, the threshold value Vth for the first overall assessment value V1 and the threshold value Vth for the second overall assessment value V2 are illustrated as being equal to each other, for example, 15 points. However, the threshold value Vth for the first overall assessment value V1 and the threshold value Vth for the second overall assessment value V2 may be different from each other.

In a case where the gradients of the approximate formulas F1 and F2 are negative, for example, in a case where the heart failure does not tend to be exacerbated, the predicting section 215 may not calculate timings at which the first overall assessment value V1 and the second overall assessment value V2 will reach the threshold value Vth.

Next, the indicating section 216 will be described below.

The indicating section 216 indicates information representing the predicted transitions of the overall assessment values V1 and V2 from the predicting section 215 to the patient P and the doctors A and B each as the user. According to the present exemplary embodiment, the indicating section 216 changes the contents to be indicated depending on the user.

In a case where the user is the patient P, the indicating section 216 indicates at least an earlier one of the first timing t3 and the second timing t4. Furthermore, the indicating section 216 indicates a recommended action depending on the period from the time at which the indication is made to the earlier one of the first timing t3 and the second timing t4. For example, if the number of days from the time at which the indication is made to the earlier timing is one or less, for example, in a case where the risk to the patient P is higher, for example, then the indicating section 216 indicates to the patient P that the patient P should immediately be examined at the medical organization to which the heart-failure specialist A belongs. Moreover, if the number of days from the time at which the indication is made to the earlier timing is three or less, for example, then the indicating section 216 indicates to the patient P that the patient P should be examined until next day at the medical organization to which the regular doctor B belongs. Moreover, if the number of days from the time at which the indication is made to the earlier timing is seven or less, for example, then the indicating section 216 indicates to the patient P that the patient P should be examined within one week at the medical organization to which the regular doctor B belongs. Moreover, if the number of days from the time at which the indication is made to the earlier timing exceeds seven and the earlier timing is after a scheduled hospital visiting date, then the indicating section 216 indicates to the patient P that the patient P should visit the medical organization to which the regular doctor B belongs, as scheduled.

In a case where the user is the general internist B, the indicating section 216 indicates at least an earlier one of the first timing t3 and the second timing t4. Furthermore, the indicating section 216 may provide the general internist B with the chronological data of the first overall assessment value V1, the chronological data of the second overall assessment value V2, and a graph, for example, the graph illustrated in FIG. 6, plotting approximate curves based on the approximate formulas F1, F2, etc., the accuracy of approximation, etc.

In a case where the user is the heart-failure specialist A, the indicating section 216 may provide a graph plotting the chronological data D1 and D2, for example, measured data, etc.

The way in which the indicating section 216 indicates the data and other information is not limited to any particular process. The indicating section 216 may display the data and other information regarding a display unit of the control unit 130 of the measuring unit 100 and display units of the operating terminals 310 and 320 of the doctors A and B, may transmit the data and other information as speech, or may send the data and other information with e-mails to the doctors A and B, for example.

The indicating section 216 may not change the contents to be indicated depending on the user. For example, the indicating section 216 may indicate an earlier one of the first timing t3 and the second timing t4 to all of the patient P and the doctors A and B. Furthermore, for example, the indicating section 216 may provide all of the patient P and the doctors A and B with the chronological data of the first overall assessment value V1, the chronological data of the second overall assessment value V2, and a graph, for example, the graph illustrated in FIG. 6, plotting approximate curves based on the approximate formulas F1, F2, etc., the accuracy of approximation, etc. Moreover, the indicating section 216 may indicate a recommended action to not only the patient P, but also the doctors A and B.

Assisting Method

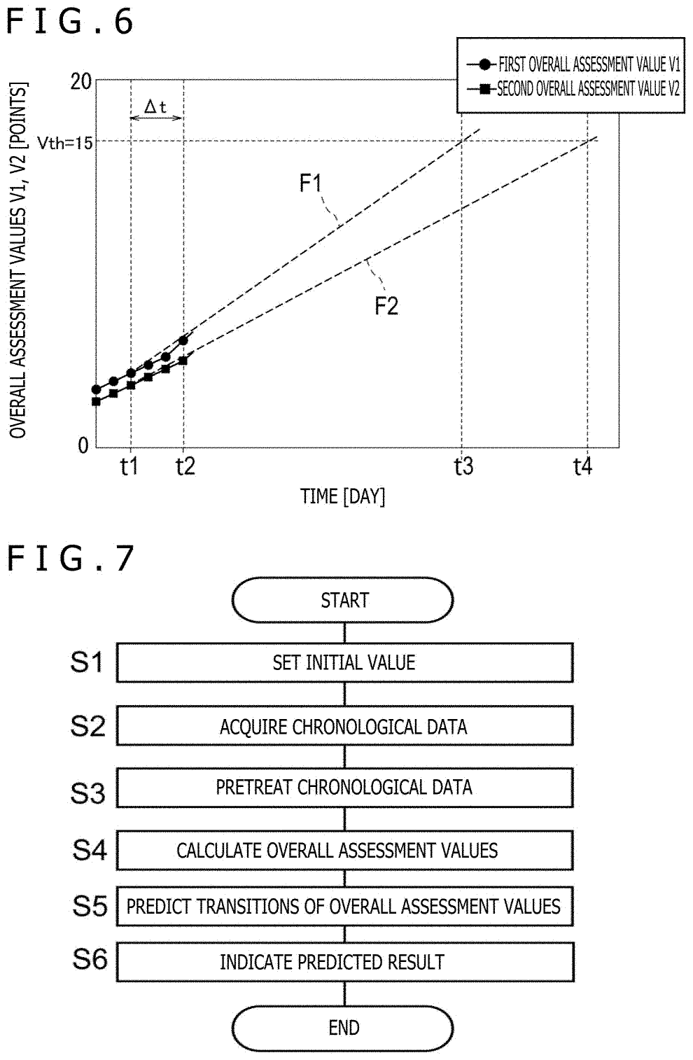

Next, an assisting method according to the present exemplary embodiment will be described below. FIG. 7 is a flowchart of the assisting method according to the present exemplary embodiment. In an example to be described below, it is assumed that the patient P is discharged from the medical organization to which the specialist A belongs, and the general internist B at a clinic does a follow-up on the patient P who has been discharged and refers the patient P to the specialist A for treatment depending on the condition of the patient P.

The assisting method according to the present embodiment will generally be described below with reference to FIG. 7. In the assisting method, an initial value for an amount of congestion is set (setting step S1), and chronological data D1 of amounts of congestion in at least a portion of the body of the patient P and chronological data D2 of a blood flow rate in the limb of the patient P are acquired (data acquiring step S2). The acquired chronological data D1 and D2 are pretreated (data processing step S3), and overall assessment values V1 and V2 are calculated (assessing step S4). Transitions of the overall assessment values V1 and V2 are predicted (predicting step S5), and information according to the predicted result is indicated to the users (indicating step S6). These steps will be described in detail below.

First, the setting step S1 will be described below. The setting step S1 is carried out on the day when the patient P is discharged from the medical organization to which the specialist A belongs, for example.

On the day of discharge, the patient P has the measuring units 110 and 120 attached to its body. Thereafter, the measuring unit 100 measures an amount of pulmonary congestion, an amount of systemic congestion, and a blood flow rage in the limb at time intervals ranging from every one minute to every one hour, for example. However, in a case where the measuring units 110 and 120 are detached from the body of the patient P, the measuring unit 100 may interrupt its measurements.

The initial value setting section 212 prompts the specialist A to specify initial values for amounts of congestion. In a case where the patient P has completely recovered from systemic congestion and pulmonary congestion, the specialist A specifies 0 (zero) for amounts of congestion. In a case where the patient P has not sufficiently recovered from systemic congestion and pulmonary congestion, the specialist A specifies predetermined values depending on the levels of the amounts of congestion as initial values for amounts of congestion. The initial value setting section 212 sets initial values for amounts of congestion on the basis of the specified values.

Next, the data acquiring step S2 through the predicting step S5 will be described below. The data acquiring step S2 through the predicting step S5 are carried out after the patient P has been discharged from the hospital.

First, the data acquiring step S2 will be described below.

The data acquiring section 211 acquires from the measuring unit 100 chronological data D1 and D2 at a predetermined timing. The timing at which the data acquiring section 211 acquires the chronological data D1 and D2 from the measuring unit 100 is not limited to any particular timing. The data acquiring section 211 can acquire chronological data D1 and D2 from the measuring unit 100 once a day or at a timing when at least one of the patient P and the doctors A and B as the users gives a request to predict transitions of the overall assessment values V1 and V2, for example.

Next, the data processing step S3 will be described below.

The data processing step S3 pretreats the chronological data D1 and D2.

First, as illustrated in FIG. 5B, the data processing section 213 calculates an average value of the chronological data D1 of the amounts of congestion acquired on the day of discharge, for example, an initial average value of the chronological data D1 of the amounts of congestion. Next, the data processing section 213 calculates a value, for example, an offset value of the chronological data D1 of the amounts of congestion, by subtracting the initial average value of the chronological data D1 of the amounts of congestion from the chronological data D1 of the amounts of congestion acquired after the patient P has been discharged from the hospital, and adding the initial values for the amounts of congestion set by the initial value setting section 212 to the difference. Next, the data processing section 213 calculates an average value, for example, an average value of the chronological data D1 of the amounts of congestion, of offset values of the chronological data D1 of the amounts of congestion acquired per predetermined period, for example, per day.

Next, the data processing section 213 calculates an average value of the chronological data D2 of the temperature of the limb acquired per predetermined period, for example, per day.

The data processing step S3 may be carried out at a predetermined timing, for example, once a day, or may be carried out at a timing when the patient P and the doctors A and B as the users give a request to predict overall assessment values V1 and V2. The sequence in which to pretreat the chronological data D1 and D2 is not limited to the above order. The chronological data D2 of the blood flow rate in the limb may be pretreated first, for example. The chronological data D1 and D2 may be pretreated concurrent with each other.

Next, the assessing step S4 will be described below.

First, the assessing section 214 chronologically calculates the pulmonary congestion assessment value C11 depending on the level of pulmonary congestion, using the average value of the chronological data D11 of the amount of pulmonary congestion.

Next, the assessing section 214 chronologically calculates the systemic congestion assessment value C12 depending on the level of systemic congestion, using the average value of the chronological data D12 of the amount of systemic congestion.

Then, the assessing section 214 chronologically calculates the blood flow assessment value C2 depending on the level of a blood flow rate in the limb, using the average value of the chronological data D2 of the blood flow rate in the limb.

Next, the assessing section 214 substitutes the calculated pulmonary congestion assessment value C11, the calculated systemic congestion assessment value C12, and the calculated blood flow evaluation value C2 for those in the equations 3 and 4, thereby calculating the first overall assessment value V1 depending on the levels of the amount of pulmonary congestion and the blood flow rate in the limb and the second overall assessment value V2 depending on the levels of the amount of systemic congestion and the blood flow rate in the limb.

The assessing step S4 may be carried out at a predetermined timing, for example, once a day, or may be carried out at a timing when at least one of the patient P and the doctors A and B as the users gives a request to predict overall assessment values V1 and V2. The sequence in which to calculate the first overall assessment value V1 and the second overall assessment value V2 is not limited to the above order. The second overall assessment value V2 may be calculated first, for example. The first overall assessment value V1 and the second overall assessment value V2 may be calculated concurrent with each other.

Next, the predicting step S5 will be described below.

First, as illustrated in FIG. 6, the predicting section 215 determines the first approximate formula F1 that approximates the chronological data of the first overall assessment value V1 acquired during the period (t1 through t2) that goes back the predetermined period Δt from the latest measurement time t2 for the chronological data D1 and D2. At this time, the predicting section 215 calculates the accuracy of the determined first approximate formula F1.

Next, the predicting section 215 determines the second approximate formula F2 that approximates the chronological data of the second overall assessment value V2 acquired during the period (t1 through t2) that goes back the predetermined period Δt from the latest measurement time t2 for the chronological data D1 and D2. At this time, the predicting section 215 calculates the accuracy of the determined second approximate formula F2.

Next, the predicting section 215 calculates the first timing t3 at which the first overall assessment value V1 will reach a threshold value and the second timing t4 at which the second overall assessment value V2 will reach a threshold value, using the first approximate formula F1 and the second approximate formula F2.

The predicting step S5 may be carried out at predetermined timings, for example, four times a day, or may be carried out at a timing when at least one of the patient P and the doctors A and B as the users gives a request to predict overall assessment values V1 and V2. The sequence in which to determine the first approximate formula F1 and the second approximate formula F2 is not limited to the above order. The second approximate formula F2 may be calculated first, for example. The first approximate formula F1 and the second approximate formula F2 may be determined concurrent with each other.

Next, the indicating step S6 will be described below.

In a case where the user is the patient P, the indicating section 216 indicates an earlier one of the first timing t3 and the second timing t4 and a recommended action depending on the period from the time at which the indication is made to the earlier timing. Therefore, the patient P can take an appropriate action depending on the exacerbation of the heart failure predicted in the future.

In a case where the user is the general internist B, the indicating section 216 indicates at least an earlier one of the first timing t3 and the second timing t4 and the chronological data of the first overall assessment value V1, the chronological data of the second overall assessment value V2, and a graph plotting approximate curves based on the approximate formulas F1, F2, etc., the accuracy of approximation, etc. The assisting system 10 is thus able to provide the general internist B who is less experienced in examining heart failures, than the heart-failure specialist A, with information that is effective to predict the exacerbation of the heart failure of the patient P. Therefore, the general internist B can treat the patient P and refer the patient P to the specialist A for treatment at a stage earlier and able to avoiding the heart failure of the patient P becoming greatly exacerbated.

In a case where the user is the heart-failure specialist A, the indicating section 216 can provide a graph plotting the chronological data D1 and D2, for example, measured data, etc. Therefore, the heart-failure specialist A can rather easily grasp the disease state of the patient P and can rather easily determine whether or not the patient P should be hospitalized, and/or which of the left cardiac failure and the right cardiac failure should preferentially be treated, etc., for example.

The indicating step S6 may be carried out at predetermined timings, for example, four times a day, or may be carried out at a timing when at least one of the patient P and the doctors A and B as the users gives a request to predict overall assessment values V1 and V2.

The assisting method according to the present exemplary embodiment has been described above. However, the assisting method is not limited to the details described above. The measuring unit 100 may not start measurements on the day of discharge when the patient P is discharged from the medical organization to which the specialist A belongs. The measuring unit 100 may start measurements on the day when the patient P is examined at the clinic to which the doctor B belongs. In this case, steps S1 through S6 are carried out regarding the day when the patient P is examined as the day when the measuring unit 100 starts measurements. Furthermore, steps S1 through S6 may be carried out repeatedly at predetermined timings.

As described above, the assisting system 10 according to the present exemplary embodiment is an assisting system for assisting in predicting the exacerbation of a heart failure. The assisting system 10 has the data acquiring section 211 for acquiring the chronological data D1 of amounts of congestion in at least a portion of the body of the patient P and the chronological data D2 of a blood flow rate in the limb of the patient P, the assessing section 214 for chronologically calculating the overall assessment values V1 and V2 depending on the levels of the amounts of congestion and the blood flow rate in the limb, using the chronological data D1 of the amounts of congestion and the chronological data D2 of the blood flow rate, and the predicting section 215 for predicting transitions of the overall assessment values V1 and V2 using the chronologically calculated overall assessment values V1 and V2.

The assisting system 10 calculates the overall assessment values V1 and V2 representing an overall assessment of the level of congestion and the level of the blood flow rate of the patient according to Nohria-Stevenson classification, and predicts the transitions of the overall assessment values V1 and V2 using the calculated overall assessment values. Therefore, the assisting system 10 is capable of assisting in the exacerbation of a heart failure on the basis of Nohria-Stevenson classification.

The chronological data D1 of amounts of congestion include the chronological data D11 of the amount of pulmonary congestion and the chronological data D12 of the amount of systemic congestion. The assessing section 214 chronologically calculates the first overall assessment value V1 depending on the levels of the amount of pulmonary congestion and the blood flow rate in the limb and the second overall assessment value V2 depending on the levels of the amount of systemic congestion and the blood flow rate in the limb. The predicting section 215 predicts the transitions of the first overall assessment value V1 and the second overall assessment value V2, using the chronologically calculated first overall assessment value V1 and the chronologically calculated second overall assessment value V2. Therefore, the assisting system 10 is capable of predicting both the exacerbation of the left cardiac failure and the exacerbation of the right cardiac failure.

Moreover, the predicting section 215 predicts the timings t3 and t4 at which the overall assessment values V1 and V2 reach the threshold value Vth. The assisting system 10 has the indicating section 216 for indicating to the user the timings t3 and t4 at which the overall assessment values V1 and V2 reach the threshold value Vth. Therefore, the user can grasp timings t3 and t4 at which the overall assessment values V1 and V2 reach the threshold value Vth.

Furthermore, the predicting section 215 predicts the first timing t3 at which the first overall assessment value V1 reaches the threshold value Vth and the second timing t4 at which the second overall assessment value V2 reaches the threshold value Vth. The assisting system 10 has the indicating section 216 for indicating to the user an earlier one of the first timing t3 and the second timing t4. Therefore, the user can grasp an earlier one of the first timing t3 and the second timing t4 and can rather easily determine which of the left cardiac failure and the right cardiac failure should preferentially be treated.

Moreover, the indicating section 216 indicates a recommended action to the user depending on the periods from the time at which the indication is made to the timings t3 and t4. Therefore, the user can take an appropriate action depending on the periods up to the timings t3 and t4.

Furthermore, the predicting section 215 determines the approximate formulas F1 and F2 that approximate the chronological overall assessment values V1 and V2. The assisting system 10 is thus simply able to predict the transitions of the overall assessment values V1 and V2.

Moreover, the assisting system 10 has the indicating section 216 that indicates to the user the level of accuracy of approximation of the approximate formulas F1 and F2. Consequently, the user can grasp the validity of the predicted results from the assisting system 10.

Moreover, the assessing section 214 calculates the congestion assessment value C1 depending on the levels of the amounts of congestion using the chronological data D1 of the amounts of congestion, and calculates the blood flow assessment value C2 depending on the level of the blood flow rate in the limb using the chronological data D2 of the blood flow rate in the limb. The predicting section 215 calculates a value representing the sum of the congestion assessment value C1 and the blood flow assessment value C2 as the overall assessment values V1 and V2. Therefore, the assisting system 10 can assist in predicting the exacerbation of the heart failure predicted in the future using the overall assessment values V1 and V2 representing an overall assessment of the amounts of congestion and the blood flow rate according to Nohria-Stevenson classification.

The chronological data D2 of the blood flow rate can include the chronological data of the temperature of the limb of the patient P and/or the chronological data of the color of the limb of the patient P. Therefore, the assisting system 10 can grasp the blood flow rate in the limb of the patient P on the basis of the chronological data of the temperature of the limb of the patient P and/or the chronological data of the color of the limb of the patient P.

The assisting method according to the present exemplary embodiment is an assisting method for assisting in predicting the exacerbation of a heart failure. The assisting method acquires the chronological data D1 of amounts of congestion in at least a portion of the body of the patient P and the chronological data D2 of a blood flow rate in the limb of the patient P, chronologically calculates the overall assessment values V1 and V2 depending on the levels of the amounts of congestion and the blood flow rate in the limb, using the chronological data D1 of the amounts of congestion and the chronological data D2 of the blood flow rate, and predicts transitions of the overall assessment values V1 and V2 using the chronologically calculated overall assessment values V1 and V2.

The assisting program according to the present embodiment is an assisting program for assisting in predicting the exacerbation of a heart failure. The assisting program executes a procedure for acquiring the chronological data D1 of amounts of congestion in at least a portion of the body of the patient P and the chronological data D2 of a blood flow rate in the limb of the patient P, a procedure for chronologically calculating the overall assessment values V1 and V2 depending on the levels of the amounts of congestion and the blood flow rate in the limb, using the chronological data D1 of the amounts of congestion and the chronological data D2 of the blood flow rate, and a procedure for predicting transitions of the overall assessment values V1 and V2 using the chronologically calculated overall assessment values V1 and V2.

The recording medium MD according to the present exemplary embodiment is a computer-readable recording medium recording the assisting program as disclosed herein.

In accordance with an exemplary embodiment, the assisting method, the assisting program, and the recording medium MD are able to predict the exacerbation of a heart failure on the basis of Nohria-Stevenson classification.

Although the exemplary embodiment of the present disclosure has been described above, the present disclosure is not limited to the details described above, but various changes and modifications may be made to the exemplary embodiment within the scope of the attached claims.

For example, the means and processes for performing various processing sequences of the assisting system may be realized by dedicated hardware circuits or a programmed computer. The assisting program may be provided on-line by way of a network such as the Internet.

The assisting system may be configured by only the server 200 according to the above exemplary embodiment, and may be used in combination with other measuring apparatus capable of measuring parameters related to an amount of congestion in at least a portion of the body of the patient and a blood flow rate in a limb of the patient (for example, the assisting system may be dispensed with the measuring unit 100).

In the above embodiment, each of the components of the server 200 is realized by a single device. However, the server configuration is not limited to such details. The server 200 may include a plurality of servers or may be virtually configured by a plurality of servers that are installed at distant locations as cloud servers.

Furthermore, a CPU of the control unit of the measuring unit may function as a data acquiring section, an assessing section, a predicting section, an indicating section, etc. Furthermore, the assisting program may be installed in each of the operating terminals of the users, enabling a CPU of each of the operating terminals of the users to function as a data acquiring section, an assessing section, a predicting section, an indicating section, etc.

Moreover, the assisting system may acquire chronological data of either one of an amount of pulmonary congestion and an amount of systemic congestion, and calculate either one of a first overall assessment value and a second overall assessment value. Furthermore, the assisting system may acquire chronological data of both an amount of pulmonary congestion and an amount of systemic congestion, and calculate either one of a first overall assessment value and a second overall assessment value.

Furthermore, the way in which an overall assessment value is calculated, for example, is not limited to the above process. For example, an overall assessment value may be a value representing the sum of a pulmonary congestion assessment value, a systemic congestion assessment value, and a blood flow assessment value. In accordance with an exemplary embodiment, an overall assessment value may be a value representing an overall assessment of the levels of an amount of pulmonary congestion, an amount of systemic congestion, and a blood flow rate in a limb.

Moreover, chronological data may not be pretreated before an overall assessment value is calculated.

Furthermore, the predicting section may not calculate a timing at which an overall assessment value reaches a threshold value, and may simply determine an approximate formula for an overall assessment value, for example. In this case, a transition of the overall assessment value in the future can be predicted using the approximate formula calculated by the predicting section.

Moreover, the assisting system may not indicate predicted results directly to the users. The assisting system may save information regarding the predicted results in a database accessible by the users for their perusal, for example.

Furthermore, the users of the assisting system may be anyone who needs to predict the exacerbation of a heart failure, and may not be limited to the patients and doctors. For example, the users of the assisting system may include nurses, pharmacists, and the like.

Moreover, the assisting system is not limited to the one used by a heart-failure specialist and a general internist at a clinic who cooperate with each other to examine patients as with the above embodiment. For example, the assisting system may be used by a plurality of specialists, for example, a vascular specialist and a heart-failure specialist, belonging to the same medical organization for examining a patient in cooperation with each other. In this case, the vascular specialist, for example, may determine when to discharge the patient depending on the predicted results about the transition of an overall assessment value. Furthermore, the assisting system is not limited to the one used in a follow-up on a patient who has suffered a heart failure after being discharged from a hospital, for example, used for prognostic control. The assisting system may be applied to patients who are highly likely to suffer a heart failure, for example.

Although the preferred embodiment of the present disclosure has been described in detail above, it should be noted that various changes and modifications may be made to the preferred embodiment of the present disclosure without departing from the scope of the appended claims.

The detailed description above describes embodiments of an assisting system, an assisting method, an assisting program, and a recording medium recording an assisting program for assisting in predicting the exacerbation of a heart failure. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An assisting system for assisting in predicting exacerbation of a heart failure, comprising:
a data acquiring section configured to acquire chronological data of amounts of congestion in at least a portion of a body of a patient and chronological data of a blood flow rate in a limb of the patient;
an assessing section configured to chronically calculate an overall assessment value depending on levels of the amounts of congestion and the blood flow rate in the limb, using the chronological data of the amounts of congestion and the chronological data of the blood flow rate; and
a predicting section configured to predict a transition of the overall assessment value using the chronologically calculated overall assessment value.

2. The assisting system according to claim 1, wherein
the chronological data of the amounts of congestion include chronological data of an amount of pulmonary congestion of the patient and chronological data of an amount of systemic congestion of the patient;
the assessing section chronologically configured to calculate a first overall assessment value depending on the levels of the amount of pulmonary congestion and the blood flow rate in the limb and a second overall assessment value depending on the levels of the amount of systemic congestion and the blood flow rate in the limb; and
the predicting section configured to predict transitions of the first overall assessment value and the second overall assessment value, using the chronologically calculated first overall assessment value and the chronologically calculated second overall assessment value.

3. The assisting system according to claim 1, wherein
the predicting section configured to predict a timing at which the overall assessment value reaches a threshold value; and
the assisting system further includes a result indicating section configured to indicate the predicted timing to a user of the assisting system.

4. The assisting system according to claim 2, wherein
the predicting section configured to predict a first timing at which the first overall assessment value reaches a threshold value and a second timing at which the second overall assessment value reaches a threshold value; and
the assisting system further includes a result indicating section configured to indicate to a user of the assisting system an earlier one of the first timing and the second timing.

5. The assisting system according to claim 3, wherein the result indicating section indicates a recommended action to the patient depending on a period from a time at which the timing is indicated to the user, to the timing.

6. The assisting system according to claim 1, wherein the predicting section is configured to determine approximate formulas that approximate the chronological overall assessment values.

7. The assisting system according to claim 6, further comprising:
an accuracy indicating section configured to indicate, to a user, information regarding accuracy of approximation of the approximate formulas.

8. The assisting system according to claim 1, wherein
the assessing section is configured to calculate:
a congestion assessment value depending on the levels of the amounts of congestion using the chronological data of the amounts of congestion, and a blood flow assessment value depending on the level of the blood flow rate in the limb using the chronological data of the blood flow rate in the limb; and
a value representing a sum of the congestion assessment value and the blood flow assessment value as the overall assessment value.

9. The assisting system according to claim 1, wherein
the chronological data of the blood flow rate includes chronological data of a temperature of the limb of the patient and/or chronological data of a color of the limb of the patient.

10. An assisting method for assisting in predicting exacerbation of a heart failure, comprising:
acquiring chronological data of amounts of congestion in at least a portion of a body of a patient and chronological data of a blood flow rate in a limb of the patient;
chronologically calculating an overall assessment value depending on levels of the amounts of congestion and the blood flow rate in the limb, using the chronological data of the amounts of congestion and the chronological data of the blood flow rate; and
predicting a transition of the overall assessment value using the chronologically calculated overall assessment value.

11. The assisting method according to claim 10, wherein the chronological data of the amounts of congestion include chronological data of an amount of pulmonary congestion of the patient and chronological data of an amount of systemic congestion of the patient, the method further comprising:
calculating a first overall assessment value depending on the levels of the amount of pulmonary congestion and the blood flow rate in the limb and a second overall assessment value depending on the levels of the amount of systemic congestion and the blood flow rate in the limb; and
predicting transitions of the first overall assessment value and the second overall assessment value, using the chronologically calculated first overall assessment value and the chronologically calculated second overall assessment value.

12. The assisting method according to claim 10, further comprising:
predicting a timing at which the overall assessment value reaches a threshold value; and
indicating the predicted timing to a user of the assisting system.

13. The assisting method according to claim 11, further comprising:
predicting a first timing at which the first overall assessment value reaches a threshold value and a second timing at which the second overall assessment value reaches a threshold value; and
indicating to a user of the assisting system an earlier one of the first timing and the second timing.

14. The assisting method according to claim 12, further comprising:
indicating a recommended action to the patient depending on a period from a time at which the timing is indicated to the user, to the timing.

15. The assisting method according to claim 10, wherein the predicting section is configured to determine approximate formulas that approximate the chronological overall assessment values.

16. The assisting method according to claim 15, further comprising:
indicating, to a user, information regarding accuracy of approximation of the approximate formulas.

17. The assisting method according to claim 10, further comprising:
calculating a congestion assessment value depending on the levels of the amounts of congestion using the chronological data of the amounts of congestion, and a blood flow assessment value depending on the level of the blood flow rate in the limb using the chronological data of the blood flow rate in the limb; and
calculating a value representing a sum of the congestion assessment value and the blood flow assessment value as the overall assessment value.

18. The assisting method according to claim 10, wherein the chronological data of the blood flow rate includes chronological data of a temperature of the limb of the patient and/or chronological data of a color of the limb of the patient.

19. A non-transitory computer-readable medium for assisting in predicting exacerbation of a heart failure, the non-transitory computer-readable medium having instructions operable to cause one or more processors to perform operations comprising:
acquiring chronological data of amounts of congestion in at least a portion of a body of a patient and chronological data of a blood flow rate in a limb of the patient;
chronologically calculating an overall assessment value depending on levels of the amounts of congestion and the blood flow rate in the limb, using the chronological data of the amounts of congestion and the chronological data of the blood flow rate; and
predicting a transition of the overall assessment value using the chronologically calculated overall assessment value.

20. The non-transitory computer-readable medium according to claim 19, wherein the chronological data of the amounts of congestion include chronological data of an amount of pulmonary congestion of the patient and chronological data of an amount of systemic congestion of the patient, the operations further comprising:
calculating a first overall assessment value depending on the levels of the amount of pulmonary congestion and the blood flow rate in the limb and a second overall assessment value depending on the levels of the amount of systemic congestion and the blood flow rate in the limb; and
predicting transitions of the first overall assessment value and the second overall assessment value, using the chronologically calculated first overall assessment value and the chronologically calculated second overall assessment value.
